(19) United States
(12) Patent Application Publication
(10) Pub. No.: US 2004/0019075 A1
(43) Pub. Date: Jan. 29, 2004

(54) New formulation for the parenteral application of crobenetine

(75) Inventor: Bernd Kruss, Hochdorf (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/446,613

(22) Filed: May 28, 2003

Related U.S. Application Data
(60) Provisional application No. 60/391,902, filed on Jun. 27, 2002.

(30) Foreign Application Priority Data
May 29, 2002 (DE) DE10223784.0

(51) Int. Cl. A61K 31/473; A61K 31/045

(52) U.S. Cl. 514/295; 514/738

(57) ABSTRACT

The invention relates to a new formulation containing crobenetine or one of the pharmaceutically acceptable salts thereof for parenteral use.

### RELATED APPLICATIONS

Benefit of U.S. Provisional Application Serial No. 60/391,902, filed on Jun. 27, 2002 is hereby claimed, and said Application is herein incorporated by reference.

The invention relates to a new formulation of (−)-(1R,2″S)-2-(2″-benzyloxy)propyl-4′-hydroxy-5,9,9-trimethyl-6,7-benzomorphan (BIII 890) or one of the pharmacologically acceptable salts thereof, particularly the hydrochloride thereof, for parenteral, particularly intravenous administration, the preparation and use thereof.

The terms “BIII 890” and “active substance” always refer to the compound (−)-(1R,2″S)-2-(2″-benzyloxy)propyl-4′-hydroxy-5,9,9-trimethyl-6,7-benzomorphan of formula

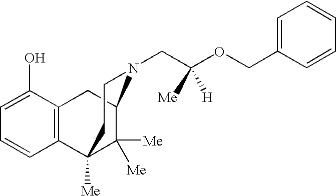

known from WO 99/14199, in the form of the free base or the corresponding acid addition salts with pharmacologically acceptable acids, particularly in the form of the hydrochloride. Other names for BIII 890 are crobenetine and [2R-[2,3(S*),6]]-1,2,3,4,5,6-hexahydro-6,11,11-trimethyl-3-[2-(phenylmethoxy)propyl]-2,6-methano- 3-benzazocin-10-ol. BIII 890 is a sodium channel blocker with neuroprotective properties; the main indications for its use are thromboembolic stroke, brain injury and pain.

The aim of the invention is to provide a new formulation for the active substance BIII 890, particularly for its hydrochloride.

The invention relates to pharmaceutical compositions for parenteral administration containing the active substance BIII 890 or one of the physiologically acceptable salts thereof, particularly the hydrochloride thereof, and mannitol as an excipient. The quantity of mannitol is preferably designed so as to produce an isotonic solution.

The pharmaceutical compositions according to the invention may optionally also contain other conventional excipients and carriers such as for example an acetic acid/acetate buffer consisting of acetic acid and sodium acetate or sodium acetate trihydrate or a citric acid/phosphate buffer consisting of, for example, citric acid and disodium hydrogen phosphate or disodium hydrogen phosphate-dihydrate. Usually, the quantities of buffer components are chosen so to produce a certain pH value and a certain buffer capacity. The solvent used is normally water for injections.

Preferably the pharmaceutical composition contains an acetic acid/acetate buffer in addition to the isotonic agent mannitol. A 0.005 to 0.05 molar, preferably a 0.005 to 0.02 molar acetic acid/acetate buffer with a pH of 3.8 to 5 is more preferred, while a 0.01 molar acetic acid/acetate buffer with a pH of 4 is most particularly preferred. The concentration specified relates to the total concentration of acetic acid and acetate together; the ratio of acetic acid to acetate results from the desired pH value. The pH specified is measured both in the pure buffer solution and also in the finished solution for injection or infusion.

Table I provides as examples the exact composition of a 0.005, 0.01 and 0.05 molar acetic acid/acetate buffer for specific pH values. The concentrations are given in mg/ml in each case and are based on 99% acetic acid (“HOAc”) and sodium acetate trihydrate (“NaOAc”).

TABLE I

| molarity | 0.005 | | 0.01 | | 0.05 | |
|---|---|---|---|---|---|---|
| pH | HOAc | NaOAc | HOAc | NaOAc | HOAc | NaOAc |
| 3.8 | 0.268 | 0.074 | 0.535 | 0.148 | 2.68 | 0.74 |
| 4.0 | 0.2505 | 0.113 | 0.501 | 0.226 | 2.505 | 1.13 |
| 4.5 | 0.18 | 0.265 | 0.36 | 0.53 | 1.8 | 2.65 |
| 5.0 | 0.095 | 0.46 | 0.19 | 0.92 | 0.95 | 4.6 |

For the indications mentioned above it is essential that the dosage is easily controlled so as to ensure that steady-state plasma levels are safely maintained. The parenteral formulation of BIII 890 according to the invention satisfies these requirements.

One embodiment according to the invention of a parenteral preparation of BIII 890 or one of the physiologically acceptable salts thereof, such as e.g. the hydrochloride, contains the active substance in doses of 1 mg/kg of body weight to 30 mg/kg of body weight per day, preferably in the range from 3-15 mg/kg of body weight. The amounts, concentrations and dosages specified relate in each case to the active substance base regardless of whether BIII 890 is used in the form of the “base” (=compound of the formula given on page 1) or in the form of one of the pharmacologically acceptable salts thereof.

The preparation is preferably administered by continuous infusion over 24 hours or optionally several days in order to maintain a steady-state plasma level. The volumes administered are in the range from 100 to 500 ml, i.e. the concentrations for administration of the active substance are in the range from 50 mg/500 ml =0.1 mg/ml (0.01%) to 1500 mg/500 ml=3 mg/ml (0.3%). A concentration of 0.03% (g/v) to 0.2% (g/v) is preferred; a concentration of 0.03% (g/v) to 0.07% (g/v) is particularly preferred.

The quantity of active substance administered can be controlled by the administration of a specific volume of one of the solutions for injection or infusion described above. For example the administration of 250 ml of a solution according to Example 3 corresponds to the administration of 175 mg of BIII 890 per day.

The following Examples are intended to illustrate the invention in more detail:

### EXAMPLES

### Example 1

Solution for infusion containing 250 mg/500 mL (“0.01 molar” acetate buffer pH 4)

| | |
|---|---|
| BIII 890 hydrochloride | 274 mg* |
| mannitol | 25000 mg |
| acetic acid 99% | 250.5 mg |
| sodium acetate trihydrate | 113.0 mg |
| water for injections ad | 500 ml |

*corresponds to 250 mg BIII 890 in the form of the base

### Example 2

Solution for infusion containing 250 mg/500 mL (“0.01 molar” acetate buffer pH 4.5)

| | |
|---|---|
| BIII 890 hydrochloride | 274 mg* |
| mannitol | 25000 mg |
| acetic acid 99% | 180.0 mg |
| sodium acetate trihydrate | 265.0 mg |
| water for injections ad | 500 ml |

*corresponds to 250 mg BIII 890 in the form of the base

### Example 3

Solution for infusion containing 350 mg/500 mL (“0.02 molar” acetate buffer pH 4)

| | |
|---|---|
| BIII 890 hydrochloride | 383.6 mg** |
| mannitol | 25000 mg |
| acetic acid 99% | 501.0 mg |
| sodium acetate trihydrate | 226.0 mg |
| water for injections ad | 500 ml |

**corresponds to 350 mg BIII 890 in the form of the base

### Example 4

solution for infusion containing 700 mg/250 mL (“0.01 molar” acetate buffer pH 4)

| | | |
|---|---|---|
| BIII 890 hydrochloride | 767 | mg*** |
| mannitol | 11000 | mg |
| acetic acid 99% | 125.25 | mg |
| sodium acetate trihydrate | 56.5 | mg |
| water for injections ad | 250 | ml |

***corresponds to 700 mg BIII 890 in the form of the base

### Example 5

Solution for infusion containing 700 mg/500 mL (“0.005 molar” acetate buffer pH 4.5)

| | |
|---|---|
| BIII 890 hydrochloride | 767 mg*** |
| mannitol | 25000 mg |
| acetic acid 99% | 90.0 mg |
| sodium acetate trihydrate | 132.5 mg |
| water for injections ad | 500 ml |

***corresponds to 700 mg BIII 890 in the form of the base

### Example 6

Solution for infusion containing 200 mg/100 mL (“0.01 molar” acetate buffer pH 4)

| | |
|---|---|
| BIII 890 hydrochloride | 219.1 mg**** |
| mannitol | 5000 mg |
| acetic acid 99% | 50.1 mg |
| sodium acetate trihydrate | 22.6 mg |
| water for injections ad | 100 ml |

****corresponds to 200 mg BIII 890 in the form of the base

We claim:

1. Pharmaceutical composition containing mannitol and BIII 890 or one of the pharmacologically acceptable salts thereof.

2. Pharmaceutical composition according to claim 1, wherein the hydrochloride of BIII 890 is used as active substance.

3. Pharmaceutical composition according to claim 1, wherein the composition additionally contains a buffer.

4. Pharmaceutical composition according to claim 3, wherein said buffer is an acetic acid/acetate buffer.

5. Pharmaceutical composition according to claim 4, wherein said acetic acid/acetate buffer has a molar concentration of between 0.005 and 0.05 and a pH of between 3.8 to 5.

6. Pharmaceutical composition according to claim 1 wherein the BIII 890 concentration is between about 0.1 mg/ml and 3 mg/ml.

7. A method for treating stroke comprised of the administration to a patient needing such treatment a therapeutically acceptable amount of a pharmaceutical composition comprised of BIII 890 or one of the pharmacologically acceptable salts thereof and mannitol.

8. The method of claim 7, wherein the pharmaceutically acceptable salt of BIII 890 is a hydrochloride salt.

9. The method of claim 7, wherein the pharmaceutical compositions are further comprised of a buffer.

10. Method according to claim 9, wherein said buffer is an acetic acid/acetate buffer.

11. Method according to claim 10, wherein the acetic acid/ acetate buffer has a molar concentration of between 0.005 and 0.05 and a pH value of between 3.8 to 5.

12. The method according to claim 7, wherein the pharmaceutical composition is administered intravenously.

13. The method of claim 7, wherein the pharmaceutical composition is administered parenterally.

14. The method according to claim 7, wherein the concentration of BIII 890 is between 0.1 mg/ml and 3 mg/ml.

15. The method of claim 14, wherein the pharmaceutical composition is further comprised of a buffer.

16. The method of claim 15, wherein the pharmaceutical composition is further comprised of an acetic acid/acetate buffer.

17. The method of claim 16, wherein the acetic acid/acetate buffer has a molar concentration of between 0.005 and 0.05 and a pH of between 3.8 to 5.
